(19) United States
(12) Patent Application Publication
(10) Pub. No.: US 2022/0041964 A1
(43) Pub. Date: Feb. 10, 2022

(54) ALGAE INCUBATOR

(71) Applicant: Josef Gundzik, Rockledge, FL (US)

(72) Inventor: Josef Gundzik, Rockledge, FL (US)

(21) Appl. No.: 17/444,438

(22) Filed: Aug. 4, 2021

Related U.S. Application Data
(60) Provisional application No. 63/060,822, filed on Aug. 4, 2020.

(51) Int. Cl. C12M 1/00; C12M 1/09; C12M 3/00

(52) U.S. Cl. CPC C12M 21/02; C12M 23/18; C12M 23/56; C12M 39/00; C12M 23/46; C12M 23/50; C12M 29/02; C12M 23/48

(57) ABSTRACT

A system for algae incubation including a reactor cell adapted to grow algae, a support buoy secured to the reactor cell, and a floating vessel secured to the reactor cell.

### RELATED APPLICATIONS

This application claims priority under 35 U.S.C. § 119(e) of U.S. Provisional Patent Application Ser. No. 63/060,822 (Attorney Docket No. 5485.00001) filed on Aug. 4, 2020 and titled ALGAE INCUBATOR. The content of that application is incorporated herein by reference.

### FIELD OF THE INVENTION

The present invention relates to systems and methods for a bioreactor. More specifically, the present invention is directed to a wave resistant photobioreactor.

### BACKGROUND OF THE INVENTION

In the fight against climate change, a great deal of time and energy have been directed toward finding renewable and environmentally friendly energy sources. However, electricity generation represents only 25% of carbon emissions with agriculture representing an additional 24%, manufacturing 21%, transportation 14%, buildings 6%, and various other sources composing the remaining 10%.

Despite investing in research, no carbon free alternatives exist for many industrial processes such as steel and plastic production. Furthermore, the engineering constraints placed on shipping and air travel make electric alternatives either economically infeasible or physically impossible with current and near future battery technology. Thus, we not only need to find an alternative source of energy, but an alternative source of carbon as well.

Biofuels represent the only solution to this problem. Current sources of biofuel require arable land that could otherwise produce food. This creates an economically disastrous opportunity cost as food is more profitable than biofuel and that is expected to remain true for the foreseeable future. Using arable land for biofuel production also presents a moral dilemma, as starvation and food insecurity affect millions of people.

Marine microalgae exist in the absence of arable, or any, land. Marine microalgae contain valuable macro and micronutrients: carbohydrates, proteins, and lipids such as DHA, Omega-3 and Omega-6. They are highly carbon efficient with 100% of their dry mass harvestable every 3 to 14 days. These characteristics make marine microalgae far more productive than current cereal crops. Furthermore, the open ocean is mostly empty and unutilized for commerce of any kind. Floating marine microalgae farms could capture some of the sunlight that falls on the 71% of the earth's surface covered by the ocean while simultaneously providing humanity with a massive source of clean energy, food, and industrial carbon.

Therefore, there exists a need of a system and method to utilize marine environments to create biofuel, food sources, and other crops.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a system for algae incubation including a reactor cell adapted to grow algae, a support buoy secured to the reactor cell, and a floating vessel secured to the reactor cell.

The reactor cell includes a first polyethylene sheet having a perimeter and a second polyethylene sheet having a perimeter secured to the perimeter of the first polyethylene sheet.

The system may further include an anchoring member and a fixed line secured to the anchoring member and the support buoy. The fixed line may be secured to the floating vessel, the reactor cell, and the support buoy.

The reactor cell may further include a percolation hose support line and an air hose. The percolation hose support line may have a first end secured to a first side of the reactor cell and a second end secured to a second side of the reactor cell. The air hose may have sidewalls with a plurality of apertures formed through an entirety of a thickness of the sidewalls along a length of the air hose secured to the percolation hose support line at a plurality of locations along the length of the air hose.

The reactor cell may yet further include a loop secured to a first side of the reactor cell and a carabineer secured to a first end of the percolation hose support line and adapted to removably secure to the loop.

The reactor cell may still further include a first hose fitting, a second hose fitting, and a hatch. The first hose fitting may be secured through a first aperture in a sidewall of the reactor cell. The second hose fitting may be secured through a second aperture in the sidewall of the reactor cell. The hatch may be formed through a third aperture in a sidewall of the reactor cell wherein the hatch is selectively positionable in a closed or an opened configuration.

The system may further include a compressor carried by the floating vessel, a valve carried by the floating vessel, a pump carried by the floating vessel, a first hose in fluid communication with the first hose fitting and the compressor, a second hose in fluid communication with the second hose fitting and the valve, and a third hose in fluid communication with the hatch and the pump.

The floating vessel may further include a compressor, a hose splitter, a first ball valve, a second ball valve, a pump, a first tank, a second tank, and a release valve. The compressor may be carried by the floating vessel. The hose splitter may have an input in fluid communication with the compressor, a first output, and a second output. The first ball valve may be in fluid communication with the first output of the hose splitter. The second ball valve may be in fluid communication with the second output of the hose splitter. The pump may be in fluid communication with the reactor cell. The first tank may be in fluid communication with the pump. The second tank may be in fluid communication with the pump. The release valve may be in fluid communication with the reactor cell.

The system may further include a fixed line secured to the reactor cell.

The floating vessel may further include a spool secured to fixed line and adapted to carry a length of the fixed line around a body of the spool.

The system may further include a weight secured along a length of the fixed line between the reactor cell and the spool.

The support buoy may include a hose fitting, a hose, and a weight. The hose fitting may be secured to a closed end of the support buoy. The hose may be secured to the hose fitting and the floating vessel. The weight may be secured to an open end of the support buoy, opposing the closed end.

The system may further include a plurality of reactor cells wherein each of the plurality of reactor cells are secured to the support buoy and the floating vessel.

### BRIEF DESCRIPTION OF THE DRAWINGS

Some embodiments of the present invention are illustrated as an example and are not limited by the FIGURES of the accompanying drawings, in which like references may indicate similar elements.

FIG. 1 is a block diagram of the algae incubation system according to an embodiment of the present invention.

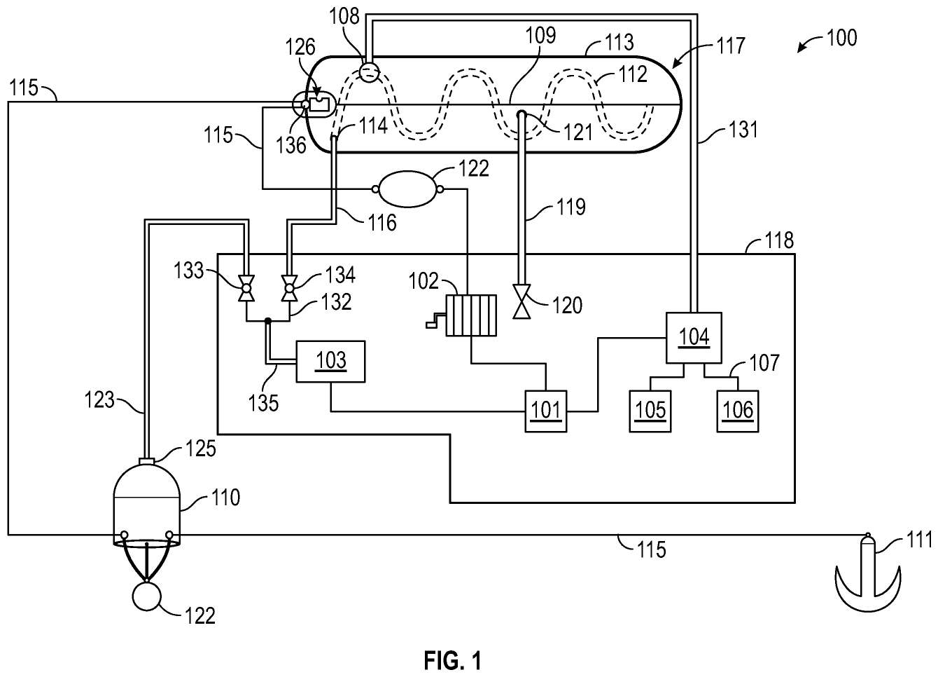

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various FIGURES and accompanying text, provides a system and method for algae incubation. The incubation system 100 may include one or more algae incubators 117 secured to both a buoy 110 and a floating vessel 118. The buoy 110 may be secured to an anchoring member 111.

The algae incubator 117 may include at least one reactor cell 113. Each reactor cell 113 may secure to a buoy 110 or a plurality of buoys 110. Each reactor cell may include a percolated air hose 112, an inbound hose fitting 114, an outbound hose fitting 121, a percolation hose support line 109 extending the length of the reactor cell 113, a carabiner 126, and a hatch 108.

In embodiments with more than one algae incubator 117, each of the algae incubators 117 may be connected to an air supply hose 116, a return hose 119, and a effluent hose 131. The air supply hose 116, return hose 119, and effluent hose 131 may be unique to each algae incubator 117 or may be shared across multiple algae incubators 117. In embodiments in which the hoses 116, 119, and 131 are shared across multiple algae incubators 117, each hose 116, 119, 131 may be used with one algae incubator 117 at a given time. Multiple algae incubators 117 may be secured to a single fixed line 115 secured to the floating vessel 118 or each algae incubator 117 may be secured to separate fixed lines 115, each of which is secured to the floating vessel 118. Similarly, each algae incubator 117 may have a separate anchoring member 111 or buoy 110 or each algae incubator 117 may share a common anchoring member 111 or buoy 110.

Each of the at least algae incubators 117 may be formed from a flexible, transparent bag forming a closed envelope. In one embodiment, by way of example, and not as a limitation, each algae incubator 117 may be formed by securing two sheets of polyethylene to one another along their perimeters. Each polyethylene sheet may have identical dimensions. The algae incubators 117 may be rectangular, circular, oblong, or the like. Each reactor cell 113 may be a separate envelope within the algae incubator 117. In embodiments having more than one reactor cell 113 within the algae incubator 117, more than one envelope may be formed in the algae incubator 117. By way of example, and not as a limitation, such a configuration may be formed by plastic welding portions of the algae incubator 117 to itself to form separate envelopes.

Each reactor cell 113 may have a bottom surface, which is positioned to be in contact with the body of water upon which the reactor cell 113 floats. Each reactor cell 113 may also have a top surface, which opposes the bottom surface. In one embodiment, a plurality of reactor cells 113 may be formed from a single bag structure. Each of the at least one reactor cells 113 may be configurable in either a closed or open position. In the closed position, the reactor cell 113 may be sealed to maintain contents within the reactor cell 113 and prevent environmental elements, such as ocean water, from entering the reactor bag 113. In the open position, contents may be placed into or removed from the interior of the reactor cell 113. The reactor cell 113 may be in a closed position when the hatch 108 is secured to the reactor cell 113 and in the open position when the hatch 108 is opened.

A fixed line 115 may secure to the algae incubator 117, floating vessel 118, and anchoring member 111. The fixed line 115 may be a single, continuous fixed line or may include one or more discrete lengths of line, collectively referred to as the fixed line 115. In one embodiment, a support buoy 110 may be secured to the fixed line 115 between the algae incubator 117 and the anchoring member 111. In one embodiment, the support buoy 110 may include a hollow cylinder, which is closed on an upper end and open on the lower end. A drop weight 122 may be secured to the support buoy 110 and positioned at the center of the open lower end so that the center of mass of the buoy 110 sits below its center of buoyancy. In one embodiment, a drop weight 122 may be secured to the support buoy 110 with three or more ropes, each secured to an aperture formed through an entirety of a thickness of the cylinder sidewalls near the lower opening. In such an embodiment, the apertures may be space equidistant from one another. In such an embodiment, an additional aperture may be formed through an entirety of a thickness of the closed upper end. A large barbed hose fitting 125 may be plastic welded to the closed end around the perimeter of the additional aperture, which may be positioned at the center on the closed upper end of the support buoy 110.

In one embodiment, a buoy hose 123 may have a first end secured to the barbed hose fitting 125 of the support buoy 110 and a second end secured to an air compressor 103 located on the floating vessel 118. In such an embodiment, the buoy hose 123 may be secured to the barbed hose fitting 125 with one or more metal clamps. The buoy hose 123 may also be secured to the fixed line 115 with metal clamps along the length of the buoy hose 123.

The second end of buoy hose 123, may be secured to a first branch of a hose splitter 132 with a metal clamp. The second branch of the hose splitter 132 may be secured to the air supply hose 116. The hose splitter 132 may be formed from PVC. The hose splitter 132 may have two branches. A first ball valve 133 may be in-line with the outlet of the first branch and a second ball valve 134 may be in-line with the outlet of the second branch. The hose splitter 132 may be in fluid communication with an output of the air compressor 103. Opening the first ball valve 133 may provide air from the air compressor 103 to the support buoy 110. Opening the second ball valve 134 may provide air from the air compressor 103 to the percolated air hose 112 of the reactor cell 113. A utility air hose 135 may have a first end secured to the hose splitter 132 and a second end secured to the compressor 103. Opening the first ball valve 133 may allow the air compressor 103 to supply air to the buoy hose 123 and the support buoy 110. Opening the second ball valve 134 may allow the air compressor 103 to supply air to the air supply hose 116 and the percolated air hose 112.

The dimensions of support buoy 110 may be selected such that when 90% of the internal volume of the support buoy 110 is filled with air and 10% of the internal volume of the support buoy 110 is filled with water, the support buoy 110 has sufficient buoyancy to remain above the waterline when under load caused by the weather affecting the floating vessel 118 and the algae incubator 117.

One or more reactor cells 113 may be formed in the algae incubator 117. Each reactor cell 113 may contain one or more percolation hose support lines 109. By way of example, and not as a limitation, the percolation hose support line 109 may be a polyethylene rope. In one embodiment, a first end of a percolation hose support line 109 may be affixed to an end of the reactor cell 113 located farthest from the floating vessel 118. This percolation hose support line 109 may be secured to in interior surface of the reactor cell 113 with a plastic weld located between the sheets that form the reactor cell 113. A carabiner 126 may be secured to the second end of the percolation hose support line 109 by a knot formed in the percolation hose support line 109. The carabiner 126 may secure to a loop of rope 136 plastic welded between the sheets that form the reactor cell 113 at the side of the reactor cell 113 nearest to the floating vessel 118. The length of the percolation hose support line 109 may be essentially equal to the length of the reactor cell 113. Such a configuration may position the length of the percolation hose support line 109 along the length of reactor cell 113.

One or more percolated air hoses 112 may be positioned within the reactor cell 113 and affixed to the percolation hose support line 109 at different locations along the length of the percolation hose support line 109. Each of the percolated air hoses 112 may have a plurality of apertures formed along its length. The plurality of apertures may be at regular intervals and may be positioned along an entirety of the length of the percolated air hose 112. Each of the percolated air hoses 112 may have an open first end secured to the hose fitting 114. The second end of the percolated air hoses 112 may be closed. A first end of the air supply hose 116 may be secured to the hose fitting 114 with a second end of the air supply hose 116 secured to a second branch of the ball valve. The ball valve may be opened to receive air from the air compressor 103. Such a configuration places the percolated air hose 112 in fluid communication with the air supply hose 116 through the hose fitting 114. The supply hose 116 may be in fluid communication with and receive a supply of air from compressor 103. The compressor 103 may be carried by the floating vessel 118. Each of the percolated air hoses 112 may secure to and be in fluid communication with a hose fitting 114 through which it maintains fluid communication with air supply hose 116. In one embodiment, fabric may cover an entirety of the exterior surface of the percolated air hose 112. The fabric may be a woven material. In such an embodiment, staples may secure the fabric to the percolated air hose 112.

An inbound hose fitting 114 having a first end and a second end may be secured to each reactor cell 113. In one embodiment, the inbound hose fitting 114 may be barbed on both ends. The first end of the inbound hose fitting 114 may secure to the top surface of each reactor cell 113. The inbound hose fitting 114 may be positioned near the side of the top surface of the reactor cell nearest to the floating vessel 118. A section of polyethylene tubing may be affixed to the first end of each hose fitting 114. The percolated air hose 112 may secure to the first end of the hose fitting 114. In embodiments in which the inbound hose fitting 114 is a barbed hose fitting, the polyethylene tubing may run only half the length of the upper part of the inner barb, located on the first end of the hose fitting 114. The lower half of the inner barb located on the first end of the hose fitting 114, may secure to percolated air hose 112 with a metal clamp. The air supply hose 116 may be secured to the second end of the inbound hose fitting 114 with a metal clamp. The section of polyethylene tubing may be secured to the barb of the inbound hose fitting 114 with a metal clamp.

An outbound hose fitting 121 having two ends may be secured to each reactor cell 113. In one embodiment, the outbound hose fitting 121 may be barbed. One end of the outbound hose fitting 121 may secure to the top surface of each reactor cell 113. The outbound hose fitting 121 may be positioned near the center point of the top surface of the reactor cell 113. A section of polyethylene tubing may be affixed to one end of each outbound hose fitting 121 between the outbound hose fitting 121 and the reactor cell 113. The section of polyethylene tubing and return hose 119 may secure to the end of the hose fitting 121 outside of reactor cell 113 with a metal clamp.

Return hose 119 may run from the outbound hose fitting to the air release valve 120. Each air release valve 120 may be carried by the floating vessel 118. The air release valve 120 may be opened to release air from the interior of the reactor cell 113 and prevent the air from pooling within the reactor cell 113 while retaining the effluent within the reactor cell 113.

In one embodiment, the air release valve 120 may include a Polyvinyl Chloride (PVC) connector bounded on its lower end by a wire mesh and on its upper end by a gasket attached to a PVC pipe. In embodiments utilizing a gasket, the gasket may secure to the inside of the PVC pipe. A ball bearing having a density less than the density of water and a diameter less than the diameter of the pipe and greater than the diameter of the inside opening of the PVC pipe may be positioned between the wire mess and the gasket. Each air release valve 120 may be positioned inside a pipe carried by the floating vessel 118 and secured to the floating vessel 118 in a vertical configuration. Such a configuration may ensure that the air release valve 120 maintains an upright position.

In one embodiment, each reactor cell 113 may include a hatch 108, which is positionable in either an open or a closed positioned. When placed in the open position, the contents of the reactor cell 113 may be accessed, placed into, or removed from the reactor cell 113. When the hatch 108 is in the closed position, the contents of the reactor cell 113 may be sealed within the reactor cell 113. The hatch 108 may be secured to or formed in the upper surface of reactor cell 113. In one embodiment, the hatch 108 may include or be formed from a standard 6-inch, Drain Waste and Vent (DWV) cleanout adapter. Such an embodiment of the hatch 108 may secure to the upper surface of reactor cell 113 by metal clamp.

An effluent pump 104 may be in fluid communication with the hatch 108 through an aperture formed in the hatch 108 and provide fertilizer and/or seed algae to the interior volume of the reactor cell 113. The effluent pump 104 may be carried by the floating vessel 118 and connected to the hatch 108 by an effluent hose 131.

The fixed line 115 may secure to a support buoy 110. The fixed line 115 may be any rope with a first end secured to a support buoy 110 and a second end secured to the reactor cell 113. In one embodiment, the anchoring member 111 may be an anchor resting on the ocean floor. In another embodiment, the anchoring member 111 may be a sea anchor. In an additional embodiment, the anchoring member 111 may be a buoy affixed to the ocean floor. In one embodiment, an end of the fixed line 115 may be secured to a spool 102. The spool 102 may be carried by a floating vessel 118. The spool 102 may be rotated to collect the fixed line 115 and all reactor cells 113 secured to the fixed line about the body of the spool 102.

A drop weight 122 may be affixed to the fixed line 115 at a point between the floating vessel 118 and the algae incubator 117. In such an embodiment, the drop weight 122 may also serve as the anchoring member 111. The drop weight 122 may be nominally carried by the floating vessel 118. In the event another vessel approaches the algae incubator 117 or if for any other reason it is desirable to position the algae incubator 117 beneath the surface of the ocean, the drop weight 122 may be thrown overboard the floating vessel 118. The weight of the drop weight 122 may sink the algae incubator 117 and support buoy 110 below the surface of the ocean. An adequate length of line 115 may be positioned between the drop weight 122 and the spool 102 to allow the drop weight 122 to sink to or near the ocean floor, bringing the algae incubator 117 with it, while remaining tethered to the floating vessel 118. Such a placement of the algae incubator 117 beneath the surface of the ocean may prevent collisions with the algae incubator 117 and other vessels. In one embodiment, the buoy hose 123 may be severed or the clamp attaching the buoy hose 123 to the first ball valve 133 may be severed to disconnect the buoy hose 123 from the air compressor 103. Such a disconnection may remove air from the support buoy 110 and facilitate sinking of the algae incubator 117 and support buoy 110.

An air supply hose 116 may have a first end secured to the inbound hose fitting 114 of the reactor cell 113 and a second end secured to an air compressor 103 or a carried by the floating vessel 118. In one embodiment, the second end of the air supply hose 116 may secure to a hose splitter 132. A ball valve may be placed inline with the air supply hose 116 and the compressor such that opening the second ball valve 134 may allow air to flow from the compressor 103 to the percolated air hose 112 and closing the ball valve 134 may prevent air from flowing from the compressor 103 to the percolated air hose 112. A return hose 119 may have a first end secured to the outbound hose fitting 121 and a second end secured to the air release valve 120 carried by the floating vessel 118.

The floating vessel 118 may include a spool 102, a compressor 103, and a water supply system. The water supply system may further include a submersible pump, a water filter, a UV water sterilizer, and an additional pump. In addition to carrying the spool 102, the floating vessel 118 may provide air to the one or more reactor cells 113 during incubation and may process algae grown in the reactor cell 113 after incubation is complete.

The method of using the algae incubator 117 as described herein may include a temporary deployment of the one or more reactor cells 113 on the surface of the ocean or other body of water. One or more floating vessels 118, which, by way of example, and not as a limitation, may be boats, ships, and other water-surface craft, may support the reactor cell 113. In one embodiment, a floating vessel 118 may be positioned a distance off shore and may fixedly secure itself in that position. In another embodiment, a floating vessel 118 may be positioned in international waters and employ a sea anchor as an anchoring member 111. In either embodiment, the method for algae incubation remains the same.

As the anchoring member 111 is deployed to an operational depth, the support buoy 110 is affixed to the fixed line 115 or positioned at the water's surface. The reactor cells 113 are then deployed from the spool 102 by releasing additional length of the fixed line 115 into the body of water. Some length of the fixed line 115 may remain on the spool 102 so that the side of the algae incubator 117 closest to the floating vessel 118 is held above the waterline. The hatch 108 may be accessible from either the floating vessel 118 or other watercraft from this raised position. The hatch 108 may be opened and each reactor cell 113 may be filled with a cleaning solution using the effluent pump 104 and utility line 107 from cleaning tank 106. In one embodiment, the cleaning solution may be a combination of diluted citric acid and sterilized water. The cleaning solution may stay in the interior of the reactor cell 113 for 10 minutes. At the conclusion of that time, utility line 108 may lower into the bottom of reactor cell 113 and the cleaning solution retrieved by the effluent pump 104 into cleaning tank 106.

Percolated air hose 112 may be placed into reactor cell 113 by attaching it to the support line 109 at regular intervals with one or more reusable cable ties affixed to percolated air hose 112. In one embodiment, two reusable cable ties may be utilized. A cable tie around the percolated air hose 112 should be relatively tight so that the percolated air hose 112 remains fixed within the reusable cable tie, and the reusable cable tie around the support lines 109 should be relatively loose so that percolated air hose 112 may slide down the support line 109. In one embodiment, the carabiner 126 may hook onto an additional line on the floating vessel 118 so that the percolated air hose 112 may affix to additional line on the floating vessel 118 and be delivered to the support line 109 and into the reactor cell 113. These cable ties may interlock and be affixed to the percolated air hose 112 at regular intervals so that once inside reactor cell 113 the percolated air hose 112 covers the majority of the lateral plane corresponding to the inner, lower surface of reactor cell 113. Once inside the reactor cell 113, the end of percolated air hose 112 nearest to the hatch 108 may be affixed to the exposed inner barb of hose fitting 114 with a metal clamp. The carabiner 126 may be secured to the side of the reactor cell 113 at a hook formed within the cavity of the reactor cell 113.

The effluent pump 104 may then be connect to effluent tank 105 and effluent may be provided to the reactor cell 113 through the effluent hose 131, which may be positioned through the hatch 108. The effluent may include minerals, vitamins, water and live algae culture. Once enough effluent to fill the reactor cell 113 at least 5 cm deep is inside the reactor cell 113, the effluent pump 104 may be shut off.

The effluent hose 131 may be removed from the hatch 108, the hatch 108 may be closed, spool 102 may unwind so that algae incubator 117 becomes fully deployed on the water's surface and the compressor 103 may be activated to place air into the reactor cell 113 through the percolated air hose 112. Sufficient air should be provided to the interior of the reactor cell 113 to cause the reactor cell 113 to rise to the surface of the water while creating only minimal air pockets within the reactor cell 113. If too much air pools within the reactor cell 113, the compressor 103 should be deactivated and the return hoses 119 and valves 120 should be inspected for obstructions or kinks. The air compressor 103 should be run for the duration of the growth period.

Once the algae reaches a concentration sufficient to end the growth cycle or surface conditions become unfavorable, the hatch 108 may be opened and the carabiner 126 may be unhooked from its securing loop within the reactor cell 113. The percolated air hose 112 may be unfixed from hose fitting 114 and pulled off support hose 109, back into the effluent tank 105. The inner surface of the reactor cell 113 may then be scraped with a long brush through hatch 108 and the effluent pumped back into the effluent tank with the effluent pump 104.

The reactor cell 113 may be cleaned at the conclusion of the growth cycle in the same manner it was cleaned prior to starting the growth cycle. The anchoring member 111 may be recovered and stowed on the floating vessel 118 prior to the floating vessel 118 returning to port.

In one embodiment, the seawater may be processed into effluent and released after the algae is harvested. In such an embodiment, effluent storage tanks may be included in the design.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

What is claimed is:

1. A system for algae incubation comprising:
a reactor cell adapted to grow algae;
a support buoy secured to the reactor cell; and
a floating vessel secured to the reactor cell.

2. The system of claim 1 wherein the reactor cell comprises:
a first polyethylene sheet having a perimeter; and
a second polyethylene sheet having a perimeter secured to the perimeter of the first polyethylene sheet.

3. The system of claim 1 further comprising:
an anchoring member; and
a fixed line secured to the anchoring member and the support buoy.

4. The system of claim 1 further comprising:
a fixed line secured to the floating vessel, the reactor cell, and the support buoy.

5. The system of claim 1 wherein the reactor cell further comprises:
a percolation hose support line having a first end secured to a first side of the reactor cell and a second end secured to a second side of the reactor cell; and
an air hose having sidewalls with a plurality of apertures formed through an entirety of a thickness of the sidewalls along a length of the air hose secured to the percolation hose support line at a plurality of locations along the length of the air hose.

6. The system of claim 4 wherein the reactor cell further comprises:
a loop secured to a first side of the reactor cell; and
a carabineer secured to a first end of the percolation hose support line and adapted to removably secure to the loop.

7. The system of claim 1 wherein the reactor cell further comprises:
a first hose fitting secured through a first aperture in a sidewall of the reactor cell;
a second hose fitting secured through a second aperture in the sidewall of the reactor cell; and
a hatch formed through a third aperture in a sidewall of the reactor cell wherein the hatch is selectively positionable in a closed or an opened configuration.

8. The system of claim 5 further comprising:
a compressor carried by the floating vessel;
a valve carried by the floating vessel;
a pump carried by the floating vessel;
a first hose in fluid communication with the first hose fitting and the compressor;
a second hose in fluid communication with the second hose fitting and the valve; and
a third hose in fluid communication with the hatch and the pump.

9. The system of claim 1 wherein the floating vessel further comprises:
a compressor carried by the floating vessel;
a hose splitter having an input in fluid communication with the compressor, a first output, and a second output;
a first ball valve in fluid communication with the first output of the hose splitter;
a second ball valve in fluid communication with the second output of the hose splitter;
a pump in fluid communication with the reactor cell;
a first tank in fluid communication with the pump;
a second tank in fluid communication with the pump; and
a release valve in fluid communication with the reactor cell.

10. The system of claim 1 further comprising:
a fixed line secured to the reactor cell; and
wherein the floating vessel further comprises a spool secured to fixed line and adapted to carry a length of the fixed line around a body of the spool.

11. The system of claim 8 further comprising:
a weight secured along a length of the fixed line between the reactor cell and the spool.

12. The system of claim 1 wherein the support buoy comprises:
a hose fitting secured to a closed end of the support buoy;
a hose secured to the hose fitting and the floating vessel; and
a weight secured to an open end of the support buoy, opposing the closed end.

13. The system of claim 1 further comprising a plurality of reactor cells wherein each of the plurality of reactor cells are secured to the support buoy and the floating vessel.

14. A system for algae incubation comprising:
a plurality of reactor cells adapted to grow algae;
a support buoy secured to each of the plurality of reactor cells; and
a floating vessel secured to each of the plurality of reactor cells.

15. The system of claim 14 further comprising:
an anchoring member; and
a fixed line secured to the anchoring member and the support buoy.

16. The system of claim 14 wherein each of the plurality of reactor cells further comprises:
a percolation hose support line having a first end secured to a first side of the reactor cell and a second end secured to a second side of the reactor cell; and
an air hose having sidewalls with a plurality of apertures formed through an entirety of a thickness of the sidewalls along a length of the air hose secured to the percolation hose support line at a plurality of locations along the length of the air hose.

17. The system of claim 16 wherein each of the plurality of reactor cells further comprises:
a loop secured to a first side of the reactor cell; and
a carabineer secured to a first end of the percolation hose support line and adapted to removably secure to the loop.

18. The system of claim 14 wherein each of the plurality of reactor cells further comprises:
a first hose fitting secured through a first aperture in a sidewall of the reactor cell;
a second hose fitting secured through a second aperture in the sidewall of the reactor cell; and
a hatch formed through a third aperture in a sidewall of the reactor cell wherein the hatch is selectively positionable in a closed or an opened configuration.

19. The system of claim 18 further comprising:
a compressor carried by the floating vessel;
a valve carried by the floating vessel;
a pump carried by the floating vessel;
a first hose in fluid communication with the first hose fitting and the compressor;
a second hose in fluid communication with the second hose fitting and the valve; and
a third hose in fluid communication with the hatch and the pump.

20. A system for algae incubation comprising:
a reactor cell adapted to grow algae comprising:
a first polyethylene sheet having a perimeter,
a second polyethylene sheet having a perimeter secured to the perimeter of the first polyethylene sheet,
a percolation hose support line having a first end secured to a first side of the reactor cell and a second end secured to a second side of the reactor cell,
an air hose having sidewalls with a plurality of apertures formed through an entirety of a thickness of the sidewalls along a length of the air hose secured to the percolation hose support line at a plurality of locations along the length of the air hose,
a loop secured to a first side of the reactor cell,
a carabineer secured to a first end of the percolation hose support line and adapted to removably secure to the loop,
a first hose fitting secured through a first aperture in a sidewall of the reactor cell,
a second hose fitting secured through a second aperture in the sidewall of the reactor cell, and
a hatch formed through a third aperture in a sidewall of the reactor cell wherein the hatch is selectively positionable in a closed or an opened configuration;
a support buoy secured to the reactor cell, comprising:
a hose fitting secured to a closed end of the support buoy,
a hose secured to the hose fitting of the support buoy and the floating vessel, and
a weight secured to an open end of the support buoy, opposing the closed end;
a fixed line secured to the reactor cell and the support buoy;
a floating vessel secured to the fixed line and further comprising:
a compressor carried by the floating vessel,
a hose splitter having an input in fluid communication with the compressor, a first output, and a second output,
a first ball valve in fluid communication with the first output of the hose splitter,
a second ball valve in fluid communication with the second output of the hose splitter,
a pump in fluid communication with the reactor cell,
a first tank in fluid communication with the pump,
a second tank in fluid communication with the pump,
a release valve in fluid communication with the reactor cell, and
a spool secured to fixed line and adapted to carry a length of the fixed line around a body of the spool;
an anchoring member secured to the fixed line;
a first hose in fluid communication with the first hose fitting and the compressor;
a second hose in fluid communication with the second hose fitting and the valve;
a third hose in fluid communication with the hatch and the pump; and
a weight secured along a length of the fixed line between the reactor cell and the spool.
